(19) United States
(12) Patent Application Publication
(10) Pub. No.: US 2025/0248694 A1
(43) Pub. Date: Aug. 7, 2025

(54) DEVICE AND METHOD TO OBTAIN SYNOVIAL FLUID AND BIOFILM SAMPLES

(71) Applicants: Justin R. CHAMBERS, Morgantown, WV (US); William T. McClellan, Morgantown, WV (US); Alisina Shahi, Bellaire, TX (US); Ali Oliashirazi, Huntington, WV (US)

(72) Inventors: Justin R. CHAMBERS, Morgantown, WV (US); William T. McClellan, Morgantown, WV (US); Alisina Shahi, Bellaire, TX (US); Ali Oliashirazi, Huntington, WV (US)

(21) Appl. No.: 19/043,479

(22) Filed: Feb. 2, 2025

Related U.S. Application Data
(60) Provisional application No. 63/548,932, filed on Feb. 2, 2024.

(51) Int. Cl. A61B 10/00; A61B 10/02

(52) U.S. Cl. CPC A61B 10/0045; A61B 10/02

(57) ABSTRACT

A medical device includes a needle, a syringe, and a probe assembly respectively connected to a main body. The syringe may include a cavity in fluid communication with a bore through a shaft of the needle and is configured to draw a fluid into the cavity through the needle. The probe assembly may include a probe that is movable through the bore.

### PRIORITY

This application claims priority to U.S. provisional patent application No. 63/548,932 filed Feb. 2, 2024, the contents of which are incorporated by reference herein in their entirety.

### FIELD OF THE INVENTION

Aspects of the present invention relate generally to medical devices and methods and, more particularly, to devices and methods for obtaining samples from a patient.

### BACKGROUND

Periprosthetic joint infection (PJI) is a severe complication that can occur after joint replacement surgeries, such as hip or knee replacements. These infections are challenging to treat due to the presence of biofilms on the implant surface, making bacteria more resistant to antibiotics. PJI can be caused by various bacteria, with Staphylococcus aureus being a common culprit. Diagnosis often involves a combination of clinical assessment, laboratory markers, imaging, and microbiological cultures. Treatment typically includes surgical intervention and prolonged antibiotic therapy, with the approach depending on factors like the infection's duration, the causative organisms, and the patient's overall health. Early detection and management are crucial for better outcomes in PJI cases.

A significant challenge is the occurrence of false negatives when testing synovial fluid or tissue samples. This primarily happens due to prior antibiotic use, which can suppress the growth of bacteria, leading to negative culture results even when an infection is present. Additionally, some bacteria are difficult to culture, or the samples might not be handled optimally, further contributing to false negatives. Cultures typically yield around 30% accuracy and are countered by taking multiple cultures which can be costly and run the risk of contamination. These challenges underscore the importance of using advanced diagnostic methods to improve the detection of causative organisms in PJIs.

Testing the biofilm in periprosthetic joint infections (PJI) is a critical aspect, as biofilms can shield bacteria from both the immune system and antibiotics. Biofilm-associated bacteria exhibit different characteristics compared to their planktonic (free-floating) counterparts, making them harder to detect and eradicate. Traditional culture methods often fail to identify bacteria within biofilms. Advanced diagnostic techniques, such as sonication of the implant to disrupt the biofilm and release bacteria, and molecular methods like PCR, offer improved detection rates. Accurate identification of organisms within the biofilm is essential for effective treatment planning.

### SUMMARY

Implementations of the invention address the above-noted problems of the prior art by providing an inventive device and method to access the synovial fluid space, aspirate the synovial fluid for testing, and swab the implant surface to physically disrupt as well as collect the biofilm for additional testing, all within one needle puncture to the patient. Currently, periprosthetic joint infections are diagnosed based on synovial fluid culturing which often leads to false negatives. The ability to test the biofilm as well as the synovial fluid increases the probability of more accurate testing results. The challenges of taking samples of both synovial fluid and implant biofilms are the multiple needle punctures, open surgery or biopsies required to collect adequate samples for testing. Therefore, a novel device and method to collect both of these samples in the clinic with one access point is highly desirable.

Various embodiments are directed to a method and device that allow for the aspiration of the synovial fluid and sampling of the implant biofilm both within the same needle puncture. In embodiments, the device includes a main body which houses a syringe, a needle, a biofilm probe, probe seal, and a probe manipulator comprising a probe advancing slider. The probe can be constructed from various materials such as stainless steel and may include a section at the tip to disrupt and swab the biofilm area when actuated. In embodiments, the probe tip is designed to have a flexible tip with advanced surface features for sample collection. An example of a flexible tip would be a coil spring design similar to guitar string wire. The syringe may be connected to the body and is in fluid communication with the needle via a fluid connection conduit. In embodiments, the probe is advanced down through the needle until the swab portion of the probe extends out of the tip of the needle to access the implant biofilm (which may also be referred to as a prosthetic biofilm).

In accordance with aspects of the invention, a method includes accessing the implant and synovial fluid space with the device by inserting the needle into the patient implant area. The syringe is used to aspirate and draw synovial fluid up in the syringe. Once adequate fluid is obtained the probe is advanced through the needle to access the prosthetic biofilm (e.g., a biofilm formed on the prosthetic implant in the patient's body). The probe can be advanced back and forth to “wipe” the probe swab on the biofilm surface to disrupt and collect the biofilm. Once adequate swab has been obtained the probe is retracted back into its initial position. The device can now be withdrawn from the patient. Both the swab sample and fluid sample are contained within the main body to send off for testing. The fluid sample can be portioned out as needed before removing the needle and capping off the end. This will create a sealed fluid sample for transportation to the testing facility.

In accordance with aspects of the invention, a medical device may include a needle, a syringe, and a probe assembly respectively connected to a main body. The syringe may comprise a cavity in fluid communication with a bore through a shaft of the needle and is configured to draw a fluid into the cavity through the needle. The probe assembly may comprise a probe that is movable through the bore.

The probe may be movable between a first position in which the probe is retracted and a second position in which the probe extends outward from the needle. In the first position the probe may not extend outward from the needle. In the first position the probe may not extend through the bore. The probe assembly may comprise a manipulator connected to the probe and configured to move the probe relative to the main body.

The main body may comprise: a first fluid channel in fluid communication with the cavity; and a second fluid channel in fluid communication with the first fluid channel, wherein the probe is movable within the second fluid channel. The needle may comprise a third fluid channel in fluid communication between the second fluid channel and the bore. The medical device may further comprise a seal at an end of the second fluid channel. The probe may pass through the seal.

The probe may comprise: a first portion having a relatively smooth exterior surface; and a second portion having a relatively rough exterior surface. The second portion may be moveable through the bore to extend outward from the needle. The first portion may extend through a seal. The first portion may be connected to a manipulator usable to move the probe back and forth relative to the main body. The second portion may be configured to collect a sample of a biofilm.

In accordance with aspects of the invention, a medical device may include a main body comprising: a needle connection system configured for connecting a needle to the main body; and a syringe connection system configured for connecting a needle to the main body. The medical device may include a probe assembly respectively connected to a main body. The probe assembly may comprise: a probe that is selectively movable relative to the main body; and a manipulator connected to the probe.

The main body may comprise: a first fluid channel in fluid communication with the syringe connection system; and a second fluid channel in fluid communication with the needle connection system and the first fluid channel, wherein the probe is movable within the second fluid channel.

The probe assembly may comprise a seal that forms a terminus of the second fluid channel. The probe may extend through the seal.

Features which are described in the context of separate aspects and embodiments of the invention may be used together and/or be interchangeable. Similarly, features described in the context of a single embodiment may also be provided separately or in any suitable sub-combination.

### BRIEF DESCRIPTION OF THE SEVERAL VIEWS OF THE DRAWINGS

Aspects of the present invention are described in the detailed description which follows, in reference to the noted plurality of drawings by way of non-limiting examples of exemplary embodiments of the present invention.

FIG. 1 shows a perspective view of a device in accordance with aspects of the present invention.

FIGS. 2A and 2B show side views of the device of FIG. 1.

FIGS. 3A and 3B show top views of the device of FIG. 1.

FIGS. 4A and 4B show side section views of the device of FIG. 1.

FIG. 5 shows an exploded view of the device of FIG. 1.

FIGS. 6A, 6B, and 6C show views of an embodiment of a probe of the device of FIG. 1.

FIG. 7 shows an embodiment of a probe tip of the device of FIG. 1.

### DETAILED DESCRIPTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details in more detail than is necessary for the fundamental understanding of aspects of the present invention, the description taken with the drawings making apparent to those skilled in the art how several forms of the present invention may be embodied in practice.

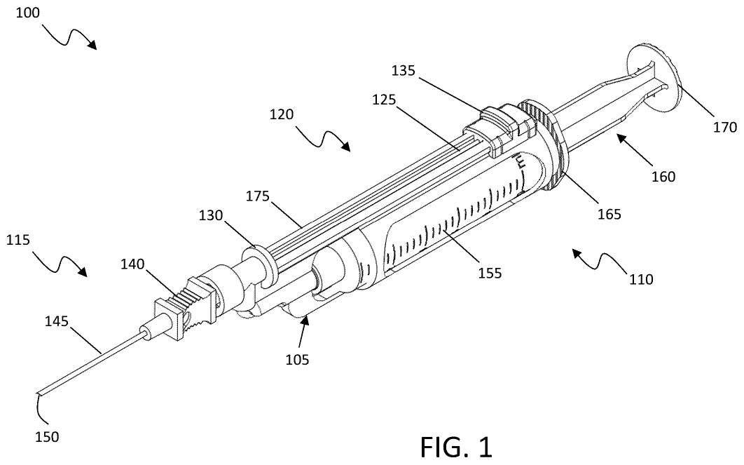

FIG. 1 shows a perspective view of a sample collection device 100 (referred to herein as “device”) in accordance with aspects of the present invention. In embodiments, the device 100 includes a main body 105 to which a syringe 110 and a needle 115 may be detachably connected (e.g., selectively connected and disconnected without damaging any of the main body 105, the syringe 110, or the needle 115). In embodiments, the syringe 110 is configured for collecting a sample of synovial fluid from a patient that has an implant (e.g., a prosthetic implant such as a hip or knee replacement that is implanted in the patient's body). In accordance with aspects of the invention, the device 100 further includes a probe assembly 120 comprising a probe 125, seal 130, and manipulator 135. In embodiments, the probe assembly 120 is configured for collecting a sample of a biofilm that is on the implant in the patient's body. In accordance with aspects of the invention, the device 100 is configured such that the syringe 110 may be used to collect a sample of synovial fluid and the probe assembly 120 may be used to collect the sample of the biofilm during one insertion of the needle 115 into the patient.

With continued reference to FIG. 1, in embodiments the needle 115 includes a needle hub 140 that connects the needle to the main body 105, a hollow needle shaft 145, and a bevel and lumen 150 at a distal end of the shaft 145. The needle hub 140 may connect to the main body 105 using any conventional or later developed connection system, including but not limited to screw threads, friction fit, or snap fit.

Still referring to FIG. 1, in embodiments the syringe 110 includes a barrel 155 and a plunger 160 configured draw fluid from the patient and hold the drawn fluid in the barrel 155. The barrel 155 may define a hollow cavity such as a cylinder. The barrel 155 may additionally have a flange 165 at one end. The plunger 160 may include a post having a thumb rest 170 at a first end and a stopper (not shown in FIG. 1) at a second end opposite the first end. The plunger 160 is configured to be positioned in the cavity defined by the barrel 155. The syringe 110 is configured such that a user may manipulate the plunger 160 using the flange and thumb rest 170 to axially slide the plunger 160 within the cavity to either expel fluid from the cavity or to draw fluid into the cavity, e.g., in a manner understood by those of ordinary skill in the art.

With even further reference to FIG. 1, in embodiments the main body 105 includes a guide 175 to which the manipulator 135 is slidably attached. Details of the probe assembly 120 and its operation are described at subsequent figures.

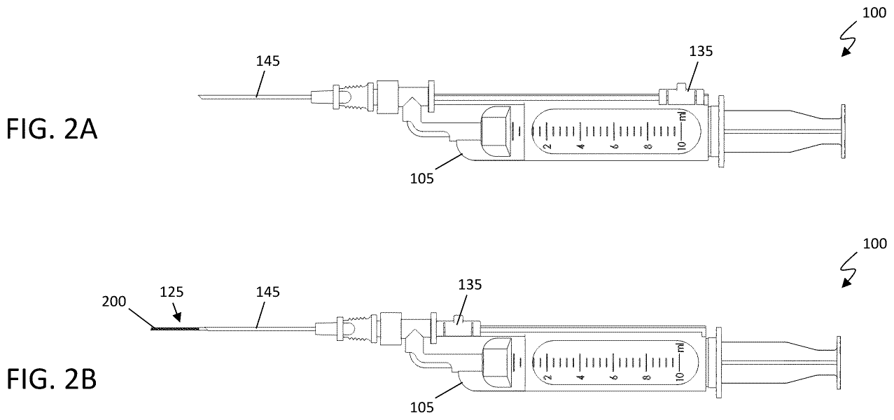

FIGS. 2A and 2B show side views of the device 100 of FIG. 1. FIG. 2A shows the device 100 in a state in which the manipulator 135 is in a first position relative to main body 105. FIG. 2B shows the device 100 in a state in which the manipulator 135 is in a second position relative to main body 105. In embodiments, when the manipulator 135 is in the second position as shown in FIG. 2B, a distal end of the probe 125, referred to herein as the probe tip 200, extends outward from the shaft 145 of the needle. In embodiments, when the manipulator 135 is in the first position as shown in FIG. 2A, the probe tip 200 is retracted and does not extend outward from the shaft 145 of the needle.

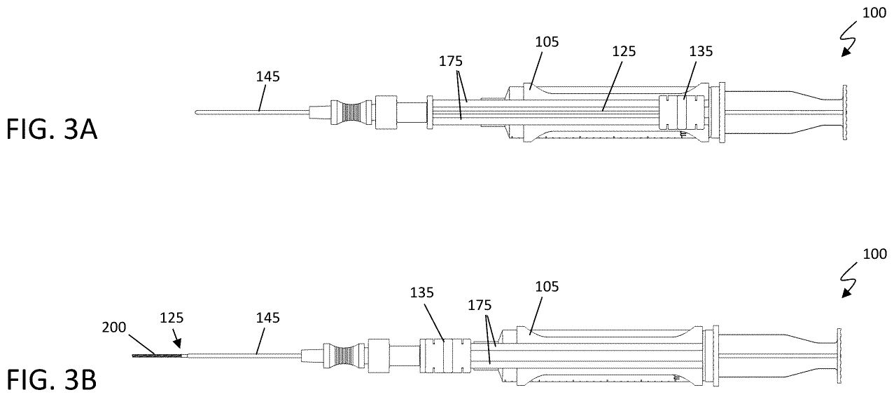

FIGS. 3A and 3B show top views of the device 100 of FIG. 1. FIG. 3A shows the manipulator 135 in the first position such that the probe tip is retracted (e.g., not extending from the needle 115. FIG. 3B shows the manipulator 135 in the second position such that the probe tip 200 extends outward from the shaft 145 of the needle 115.

With continued reference to FIGS. 3A and 3B, in embodiments the main body 105 includes the guide 175 to which the manipulator 135 is attached and along which the manipulator 135 may translate back and forth in an axial direction relative to the main body 105. In embodiments, the guide 175 comprises one or more structures (e.g., one or more ridges, one or more grooves, one or more tracks, etc.) that engage one or more corresponding structures on the manipulator 135, wherein the engagement of the one or more structures and the one or more corresponding structures is such that the manipulator 135 is connected to the main body 105 and may translate back and forth relative to the main body 105. In this manner, the manipulator 135 may be configured as a probe advancing slider.

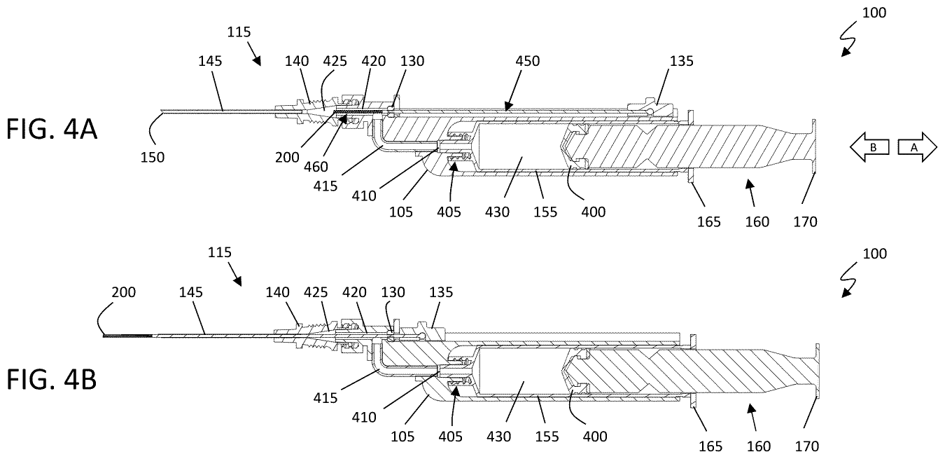

FIGS. 4A and 4B show side section views of the device 100 of FIG. 1. FIGS. 4A and 4B show an example of a stopper 400 on the plunger 160 inside the barrel 155 of the syringe. In embodiments, the barrel 155 fits inside the main body 105 and connects to the main body 105 at a syringe connection system 405. In some embodiments, the syringe connection system 405 is a screw thread, friction fit, snap fit, or other type connection system that permits the barrel 155 to be releasably connect to the main body 105. In other embodiments, the barrel 155 and the main body 105 are permanently connected, for example by molding the barrel 155 and the main body 105 together to form a unitary structure. In accordance with aspects of the present invention, when the barrel 155 is connected to the main body 105 via the syringe connection system 405, an outlet 410 at a distal end of the barrel 155 is aligned with and in fluid communication with a first fluid channel 415 inside the main body 105. In embodiments, the first fluid channel 415 is in fluid communication with a second fluid channel 420 also inside the main body 105. In embodiments, when the needle 115 is connected to the main body 105, the second fluid channel 420 is in fluid communication with a third fluid channel 425 in the needle hub 140, which is in fluid communication with a hollow bore extending through the shaft 145 of the needle 115 and ending at the bevel and lumen 150 of the needle 115. In this manner, a continuous fluid path is defined from an interior cavity 430 of the barrel 155 of the syringe to the bevel and lumen 150 at the end of the shaft 145 of the needle 115. As should be readily understood from this description, moving the plunger 160 relative to the barrel 155 in the axial direction of arrow A can be used to draw fluid into the needle 115, through the fluid channels 425, 420, and 415, and into the cavity 430 of the barrel 155. Conversely, moving the plunger 160 relative to the barrel 155 in the axial direction of arrow B can be used to expel fluid from the cavity 430 of the barrel 155, through the fluid channels 415, 320, and 425, and out through the needle 115.

With continued reference to FIGS. 4A and 4B, in embodiments the probe tip 200 is positioned in the second fluid channel 420 and/or the third fluid channel 425 when the manipulator 135 is in the first position shown in FIG. 4A. In accordance with aspects of the invention, moving the manipulator 135 from the first position shown in FIG. 4A to the second position shown in FIG. 4B causes the probe tip 200 to move through the third fluid channel 425, into and through the bore of the shaft 145, and out of the lumen at the end of the shaft 145 (e.g., from a retracted position to an extended position). In accordance with further aspects of the invention, moving the manipulator 135 from the second position shown in FIG. 4B to the first position shown in FIG. 4A causes the probe tip 200 to move from the extended position to the retracted position. In embodiments, third fluid channel 425 in the hub 140 has a funnel-like shape that guides the free end of the probe into the bore of the shaft 145.

Still referring to FIGS. 4A and 4B, in embodiments the probe 125 passes through the seal 130 of the probe assembly. In embodiments, the seal 130 creates a fluid-tight seal around the probe 125 and at an end of the second fluid passage 420, thereby forming a terminus of the second fluid passage 420. In this manner, when the plunger 160 is pulled outward to draw fluid into the cavity 430, the fluid is drawn through the lumen of the needle 115 but is not drawn through or around the seal 130.

With still further reference to FIGS. 4A and 4B, in embodiments the probe 125 comprises a first portion 450 proximate to the manipulator 135 and a second portion 460 that comprises the probe tip 200. In accordance with aspects of the invention, the second portion 460 has a relatively rough exterior surface that is configured for collecting a sample of a biofilm as described herein, while the first portion 450 has a relatively smooth exterior surface that passes through and that contacts the seal 130 to provide the fluid-tight seal at the terminus of the second fluid passage 420. In embodiments, the probe 125 is structured such that the first portion 450 contacts the seal 130 when the manipulator 135 is in the first position shown in FIG. 4A and the second position shown in FIG. 4B, and at every position in between. In this manner, the smoother surface of the first portion 450 remains in contact with the seal 130, and therefore maintains the fluid tight seal, throughout the entire stroke of the probe 125 from the retracted position to the extended position and back again.

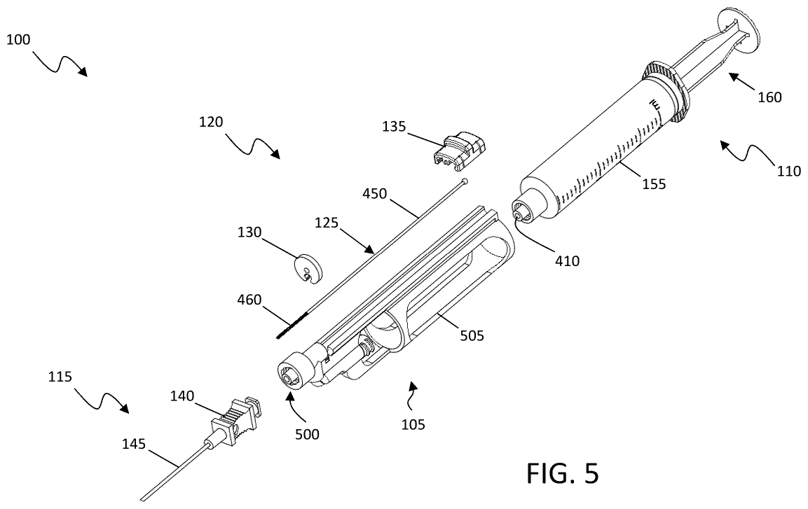

FIG. 5 shows an exploded view of the device 100 of FIG. 1 including: needle 115 with shaft 145 and hub 140 that is configured to connect to a needle connection system 500 of the main body 105; syringe 110 including barrel 155 and plunger 160, where the distal end of the barrel 155 (including outlet 410) is configured to connect to the syringe connection system 405 of the main body 105; and probe assembly 120 comprising probe 125, manipulator 135, and seal 130, where the manipulator 135 is configured to be connected to a guide 175 of the main body 105. As shown in FIG. 5, the probe 125 includes the first portion 450 and second portion 460. As shown in FIG. 5, the main body 105 may comprise a supporting structure 505 that receives the barrel 155 when the barrel 155 is connected to the main body 105. In some embodiments, the supporting structure 505 comprises a sleeve for supporting the syringe when the syringe is connected to the main body 105.

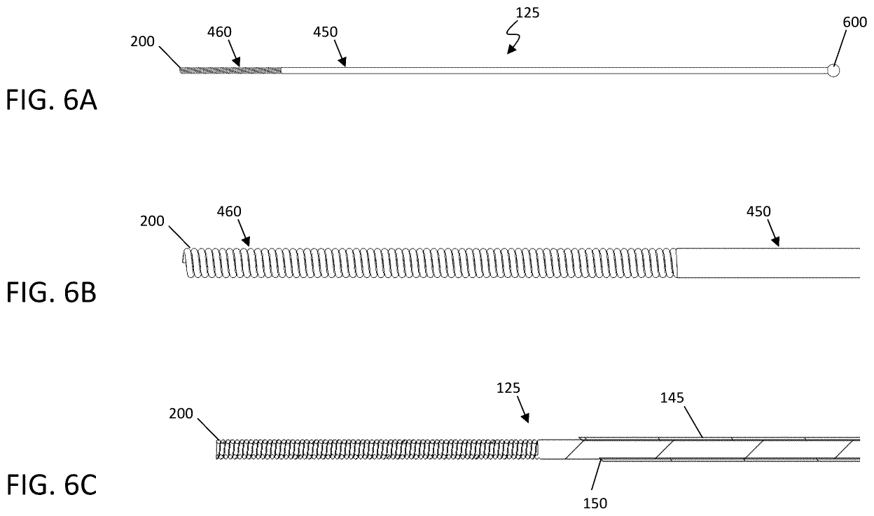

FIGS. 6A, 6B, and 6C show views of an embodiment of a probe 125 of the device 100 of FIG. 1. As shown in FIGS. 6A and 6B, and as described herein, the probe 125 may include a first portion 450 with a relatively smooth exterior surface and a second portion 460, including the probe tip 200, with a relatively rough exterior surface. The probe 125 may also include an element at an end opposite the end of the probe tip 200, the element 600 being configured to attach the probe 125 to the manipulator 135. FIG. 6C shows an example of the probe 125 relative to the shaft 145 of the needle when the manipulator is in the second position (e.g., FIGS. 2B, 3B, 4B), with the probe 125 extending through a bore in the shaft 145 and being in an extended position in which the probe tip 200 extends outward from the lumen 150 of the shaft 145.

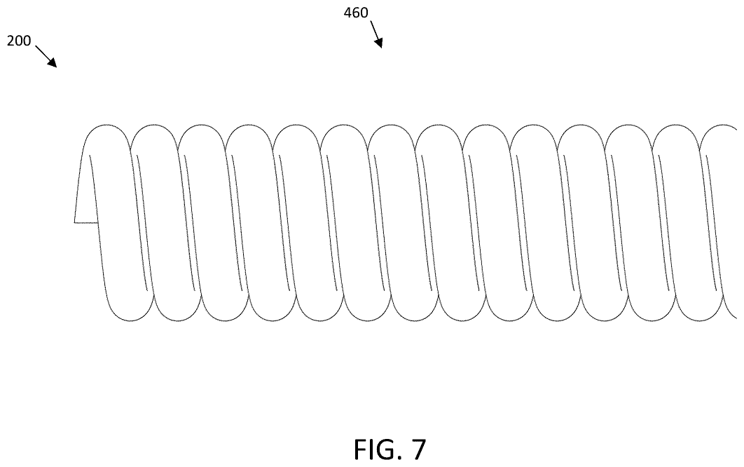

FIG. 7 shows an embodiment of a probe tip 200 of the device 100 of FIG. 1. As described herein, the probe can be constructed from various materials such as stainless steel and may include a section (e.g., second portion 460) at the tip to disrupt and swab the biofilm area when actuated. In embodiments, the probe tip 200 is designed to have a flexible tip with advanced (e.g., roughened, not smooth) surface features for sample collection. An example of a flexible tip would be a coil spring design similar to guitar string wire. In one example, the probe comprises a two piece construction including the first portion 450 composed of solid wire (e.g., stainless steel) that is relatively more rigid and less susceptible to bending and the second portion 460 composed of coiled wire that is relatively less rigid and more susceptible to bending and which has a textured surface for disrupting the biofilm and holding a portion of the biofilm. The texture may be provided by the uneven surface formed by coil of as shown in FIG. 7. The texture may additionally be provided by surface finishing such as knurling or by providing another material (e.g., plastic) to which the biofilm will adhere. The probe may be a composite structure formed of different materials. For example, the first portion may comprise metal and the second portion may comprise plastic. In another example, the first portion comprises plastic and the second portion comprises metal. In another example, the first portion comprises a first metal and the second portion comprises a second metal different than the first metal.

In accordance with aspects of the invention, a method includes assembling the device 100 by attaching the needle 115 to the main body 105 at the needle connection system 500 and attaching the syringe 110 to the main body 105 at the syringe connection system 405.

In accordance with aspects of the invention, a method includes using the device 100 to collect a fluid sample and a biofilm sample from a patient. In embodiments of the method, the probe is initially retracted (e.g., the manipulator 135 is i the first position) and the plunger 155 is depressed in the cavity 430 of the barrel 155. With the device in this configuration, the shaft 145 of the needle 115 is inserted into the patient's body such that the distal end of the shaft is adjacent the surface of the implant in the patient's body. With the device in this position (e.g., with the needle in the patient), the plunger is withdrawn relative to the barrel 155 which draws synovial fluid through the shaft 145, through the fluid passages 425, 420, 415, and into the cavity 430. With the device still in this position, the manipulator 135 is moved forward relative to the main body 105, which causes the probe tip 200 to move into, through, and out of the bore of the shaft 145. While holding the device in place relative to the patient, the manipulator 135 is moved back and forth relative to the main body 105, which causes the probe tip 200 to move back and forth relative to the shaft 145, which causes the probe tip 200 to wipe or scrape the surface of the implant to break up the biofilm on the surface of the implant. Surface texture on the probe collects sample of biofilm during this wiping/scraping. If the probe tip has a coiled configuration, the movement of the probe tip may cause bending of the coil, which creates gaps between coils that are useful for retaining the biofilm sample. After this wiping/scraping, the manipulator 135 is moved back to the first position to retract the probe back through the needle. After the probe has been retracted into the device 100, the device 100 is pulled away from the patient to extract the needle out of the patient. The method may further include removing the needle 115 from the main body 105 and fastening a cap to the needle connection system 500. The method may further include sending the capped device 100 to a lab for testing the two different samples contained in the device. In this manner, the device 100 is useable to collect two different samples from the patient using a single needle stick, which represents an improvement over current techniques.

Additional aspects of the invention include manufacturing and/or using a sample collection device described herein. Even further aspects of the invention include providing instructions for using a sample collection device described herein. The instructions may be at least one of printed and video.

It is noted that the foregoing examples have been provided merely for the purpose of explanation and are in no way to be construed as limiting of implementations of the present invention. While aspects of the present invention have been described with reference to an exemplary embodiment, it is understood that the words which have been used herein are words of description and illustration, rather than words of limitation. Changes may be made, within the purview of the appended claims, as presently stated and as amended, without departing from the scope and spirit of the present disclosure in its aspects. Although implementations of the present invention have been described herein with reference to particular means, materials and embodiments, implementations of the present invention are not intended to be limited to the particulars disclosed herein; rather, implementations of the present invention extend to all functionally equivalent structures, methods and uses, such as are within the scope of the appended claims.

What is claimed:

1. A medical device, comprising:
a needle, a syringe, and a probe assembly respectively connected to a main body;
wherein the syringe comprises a cavity in fluid communication with a bore through a shaft of the needle and is configured to draw a fluid into the cavity through the needle, and
the probe assembly comprises a probe that is movable through the bore.

2. The medical device of claim 1, wherein the probe is movable between a first position in which the probe is retracted and a second position in which the probe extends outward from the needle.

3. The medical device of claim 2, wherein in the first position the probe does not extend outward from the needle.

4. The medical device of claim 2, wherein in the first position the probe does not extend through the bore.

5. The medical device of claim 2, wherein the probe assembly comprises a manipulator connected to the probe and configured to move the probe relative to the main body.

6. The medical device of claim 1, wherein the main body comprises:
a first fluid channel in fluid communication with the cavity; and
a second fluid channel in fluid communication with the first fluid channel, wherein the probe is movable within the second fluid channel.

7. The medical device of claim 5, wherein the needle comprises a third fluid channel in fluid communication between the second fluid channel and the bore.

8. The medical device of claim 6, further comprising a seal at an end of the second fluid channel.

9. The medical device of claim 8, wherein the probe passes through the seal.

10. The medical device of claim 1, wherein the probe comprises:
a first portion having a relatively smooth exterior surface; and
a second portion having a relatively rough exterior surface.

11. The medical device of claim 10, wherein the second portion is moveable through the bore to extend outward from the needle.

12. The medical device of claim 10, wherein the first portion extends through a seal.

13. The medical device of claim 10, wherein the first portion is connected to a manipulator usable to move the probe back and forth relative to the main body.

14. The medical device of claim 10, wherein the second portion is configured to collect a sample of a biofilm.

15. A medical device, comprising:
a main body comprising: a needle connection system configured for connecting a needle to the main body; and a syringe connection system configured for connecting a needle to the main body; and
a probe assembly respectively connected to a main body, wherein the probe assembly comprises: a probe that is selectively movable relative to the main body; and a manipulator connected to the probe.

16. The medical device of claim 15, wherein the main body comprises:
a first fluid channel in fluid communication with the syringe connection system;
a second fluid channel in fluid communication with the needle connection system and the first fluid channel, wherein the probe is movable within the second fluid channel.

17. The medical device of claim 16, wherein:
the probe assembly comprises a seal that forms a terminus of the second fluid channel; and
the probe extends through the seal.
